(19) United States
(12) Patent Application Publication
(10) Pub. No.: US 2016/0354536 A1
(43) Pub. Date: Dec. 8, 2016

(54) Dose Titration Aid

(71) Applicant: NOVO NORDISK A/S, Bagsværd (DK)

(72) Inventor: Bo Radmer, Hilleroed (DK)

(73) Assignee: Novo Nordisk A/S, Bagsværd (DK)

(21) Appl. No.: 15/119,028

(22) PCT Filed: Feb. 23, 2015
(86) PCT No.: PCT/EP2015/053710

§ 371 Date: Aug. 15, 2016

(30) Foreign Application Priority Data
Feb. 24, 2014 (EP) 14156345.2

(51) Int. Cl. A61M 5/00; A61M 5/31

(52) U.S. Cl. CPC A61M 5/002; A61M 5/31; A61M 2205/583; A61M 2205/6009; A61M 2205/581; A61M 2205/582; A61M 2005/3126

(57) ABSTRACT

The present invention provides a needle arrangement (10, 20, 30) comprising a plurality of injection needles (28, 29, 39), one or more needle containers (11, 21, 22, 31, 32) for the plurality of injection needles (28, 29, 39), each of the one or more needle containers (11, 21, 22, 31, 32) being structured to hold at least one of the plurality of injection needles (28, 29, 39), and a dedication of each of the plurality of injection needles (28, 29, 39) held in the one or more needle containers (11, 21, 22, 31, 32) to one of at least two predetermined dose sizes.

### FIELD OF THE INVENTION

The present invention relates to systems for drug delivery, in particular to systems for aiding people in the self-administration of an injectable drug during a dose titration period.

### BACKGROUND OF THE INVENTION

Dose titration is often employed in the beginning of a drug treatment in order to slowly adapt the body to a particular medication and to reach a desired clinical effect. In a dose titration period the administered dose may be gradually increased over time to a predetermined target level or until a certain response is achieved.

Some dose titration schemes can be rather difficult for users to follow because they involve complex dose administration patterns. For example, if dose size X is prescribed for W1 days, dose size Y is prescribed for W2 days, dose size Z is prescribed for W3 days, and so forth, the user may easily forget which part of the period she/he is currently undergoing, i.e. the actual dose size to be administered may be mixed up with one of the other prescribed dose sizes, leading to non-compliance with the titration regimen.

DE 20 2004 000 599 U1 (Svechnikov) discloses a medical box for diabetics with rooms for appliances such as dose injectors, blood glucose measurement equipment, injection needles, and various oral medication. Some rooms are provided with descriptors which indicate the day and the time of day of use of its contents.

WO 2005/049114 (Marston) discloses a container system for hypodermic needle packs for insulin injection. The container system comprises a plate arrangement serving to indicate the time of use of a particular injection needle, and so helps the user in remembering whether a dose of insulin has been taken on a given day, or time of day.

While both of these arrangements are examples of how a user may be guided to take a dose of drug at a certain time of day they fail to provide any guidance related to which particular dose size to administer.

### SUMMARY OF THE INVENTION

It is an object of the invention to eliminate or reduce at least one drawback of the prior art, or to provide a useful alternative to prior art solutions.

In particular, it is an object of the invention to provide an arrangement which is capable of aiding a person, e.g. in the course of a dose titration period, in remembering which of a number of prescribed dose sizes to administer at a given point in time.

In the disclosure of the present invention, aspects and embodiments will be described which will address one or more of the above objects and/or which will address objects apparent from the following text.

An arrangement embodying the principles of the invention establishes a connection between an injection needle and a particular dose size. Thereby, a user opting for use of a particular injection needle will be prompted as to which dose size to administer. Further, a system may be provided wherein a predefined number of injection needles are linked to a prescribed dose size, and the mere presence of such needles serves as a direct indication of which particular part of e.g. a dose titration period the user is currently undergoing, and thus which prescribed dose size should be administered.

Accordingly, in one aspect of the invention a needle arrangement is provided comprising an injection needle, a needle container for the injection needle, and a dedication of the injection needle to a dose size. The dedication of the injection needle to a dose size may comprise a representation of the dose size, e.g. comprising a number, or a number and a unit, on the injection needle and/or on the needle container. The representation may be in the form of a visual marking printed, embossed, engraved, or otherwise graphically and/or physically applied to the injection needle and/or the needle container. Alternatively, or additionally, the representation may be tactile, e.g. in the form of braille, and/or audible. An audible representation could for example be implemented by speech synthesis, e.g. where a number, or a number and a unit, is communicated through a loudspeaker when a needle container is opened or when an injection needle is removed from a needle container.

The needle container may be a dedicated packaging suitable for enclosing a single injection needle, such as e.g. a conventional needle casing of the type having a closed first end and an open second end which is sealed by a removable seal, or it may be a reservoir adapted to contain one or more packaged or non-packaged injection needles.

In another aspect of the invention a system for administering at least two predetermined dose sizes of a liquid drug is provided, the system comprising a) dose administration means for expelling the at least two predetermined dose sizes of the liquid drug from one or more reservoirs, and b) a drug transfer arrangement comprising b1) a plurality of liquid conveying structures for sequential use with the dose administration means, b2) container means for holding the plurality of liquid conveying structures, and b3) a dedication of each of the plurality of liquid conveying structures held by the container means to one of the at least two predetermined dose sizes.

The dose administration means may be exemplified by a single drug delivery device, such as e.g. an injection device, capable of delivering each of the at least two predetermined dose sizes, or by a plurality of drug delivery devices, each being capable of delivering one or more of the at least two predetermined dose sizes.

In a further aspect of the invention a dose titration system for administering at least two predetermined dose sizes of an injectable drug is provided, the system comprising a) one or more drug delivery devices, each of the one or more drug delivery devices being capable of delivering one or more of the at least two predetermined dose sizes of the injectable drug, and b) a needle arrangement comprising b1) a plurality of injection needles for use with the one or more drug delivery devices, b2) one or more needle containers, each of the one or more needle containers being structured to hold at least one of the plurality of injection needles, and b3) a dedication of each of the plurality of injection needles held in the one or more needle containers to one of the at least two predetermined dose sizes.

With such systems every time an administration is to be performed the user will be informed of which dose size to choose when taking one of the liquid conveying structures, e.g. an injection needle, an infusion set, or the like, for connection to the dose administration means. In an ordinary dose titration phase where the dose size increases gradually the user will always opt for the liquid conveying structure corresponding to the lowest present dose size dedication.

The plurality of injection needles may be arranged in groups, where each group consists of injection needles with identical dedication. Thereby, the plurality of injection needles are arranged in groups of equal dose sizes, and it will be possible for a user to quickly identify the lowest present dose size dedication.

The plurality of injection needles may comprise a first number of injection needles corresponding to a first prescribed number of injections of a first predetermined dose size, and a second number of injection needles corresponding to a second prescribed number of injections of a second predetermined dose size. The prescribed numbers of injections of the various predetermined dose sizes may e.g. be defined by a healthcare person, such as a medical doctor, or by the manufacturer of the medicine involved in the dose titration scheme.

Such an arrangement allows for an exact predetermined transition from one titration step to the next based on a certain number of injections per titration step. Thereby, an involvement of e.g. the user's physician during the titration phase may be minimised.

The term “injection needle” as used herein is intended to also cover an injection needle assembly as known conventionally in the art, i.e. a needle cannula mounted in a hub, the hub having means for connection to a drug delivery device, the injection needle assembly either being packaged in a dedicated needle casing, e.g. of the type having a closed first end and an open second end which is sealed by a removable seal, or non-packaged.

The dedication of each of the plurality of injection needles to one of the at least two predetermined dose sizes may comprise a representation of the one of the at least two predetermined dose sizes, e.g. provided on the injection needles themselves, on the dedicated needle casings, on the needle container(s), and/or on one or more elements associated with the aforementioned. The representation may be visual, tactile, audible or otherwise perceivable by a sensory system. In particular, the representation may comprise one or more visual and/or tactile markings relating each of the plurality of injection needles to one of the at least two predetermined dose sizes by specifying the one of the at least two predetermined dose sizes. Such specification may be in the form of a numeral and possibly also a unit, so the user is able to read the dose size directly off the particular entity.

Each of the one or more drug delivery devices may comprise either a single-shot or multi-shot fixed dose device configured to deliver one particular dose size, or a single-shot or multi-shot variable dose device configured to deliver a dose size selectable from a range of dose sizes. Regardless of which the particular drug delivery device may comprise a dose display allowing the user to visually verify a set dose being ready for administration. The dose display may be mechanical, e.g. displaying characters on a scale drum or an odometer, or electronic, e.g. displaying digits or other characters in a manner similar to what is known from digital electronic watches.

Examples of drug delivery devices suitable for use in systems as described in the above are FlexPen®, FlexTouch® and NovoPen® 5, all manufactured by Novo Nordisk, and examples of injection needles suitable for use in needle arrangements for these systems are NovoTwist® and NovoFine® Plus, both marketed by Novo Nordisk.

In an exemplary embodiment of the invention the representation of one of the at least two predetermined dose sizes comprises a visual replica of a dose display of the one or more drug delivery devices.

The one or more needle containers may comprise a plurality of predefined positions for the plurality of injection needles. Each of the plurality of predefined positions may be adapted to hold exactly one injection needle and may be associated with a dose related marking, e.g. provided at or next to the predefined position. In this way, each injection needle may be stably positioned in or on a needle container and when all predefined positions relating to a particular one of the at least two predetermined dose sizes are empty the user will quickly be able to see that it is time to move on to the next dose titration step.

A predefined position may e.g. be a well configured to contain one injection needle, a dedicated spot on a cardboard to which one injection needle is removably fastened, or any other like well-defined position suitable for maintaining an injection needle in a certain position relative to another injection needle.

In particular embodiments the plurality of predefined positions are arranged in arrays, where each array holds a portion of the plurality of predefined positions which corresponds to a prescribed number of injections of one of the at least two predetermined dose sizes. This enables a very fast identification of completed titration steps. The dose related markings are either provided next to each predefined position, or indicated at each array.

Also, in particular embodiments each array holds seven of the plurality of predefined positions, corresponding to dose titration phases of one week's duration using a once daily injection scheme.

The one or more needle containers may, alternatively, comprise a first box adapted to hold the first number of injection needles and comprising an indication of the first predetermined dose size, and a second box adapted to hold the second number of injection needles and comprising an indication of the second predetermined dose size. While the injection needles in those boxes may not have well-defined positions the user will still easily see from an empty box that it is time to move on to the next dose titration step.

The one or more needle containers may further comprise an indication of a sequence for administration of the at least two predetermined dose sizes. Such indication could for example be a simple numbering or other graphical marking, e.g. provided on an exterior surface of the individual needle container. This would guide the user even further towards choosing the correct injection needle and thereby the correct dose size.

In a further aspect of the invention a needle arrangement is provided for use in a system according to any of the above described other aspects of the invention.

Specifically, a needle arrangement may be provided comprising a) a plurality of injection needles for use with one or more drug delivery devices, each of the one or more drug delivery devices being capable of delivering one or more predetermined dose sizes of a contained drug, b) one or more needle containers, each of the one or more needle containers being structured to hold at least one of the plurality of injection needles, and c) a dedication of each of the plurality of injection needles held in the one or more needle containers to one of the one or more predetermined dose sizes. The dedication may comprise a visual, audible, tactile and/or any other perceivable representation of the one of the at least two predetermined dose sizes in accordance with the above described.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 shows a system for administration of at least two predetermined dose sizes of an injectable drug according to a first embodiment of the invention,

FIG. 2 shows a system for administration of at least two predetermined dose sizes of an injectable drug according to a second embodiment of the invention, and

FIG. 3 shows a needle arrangement for use in a system for administration of at least two predetermined dose sizes of an injectable drug.

In the figures like structures are mainly identified by like reference numerals.

### DESCRIPTION OF EXEMPLARY EMBODIMENTS

When in the following relative expressions, such as “upwards” and “downwards”, are used, these refer to the appended figures and not necessarily to an actual situation of use. The shown figures are schematic representations for which reason the configuration of the different structures as well as their relative dimensions are intended to serve illustrative purposes only.

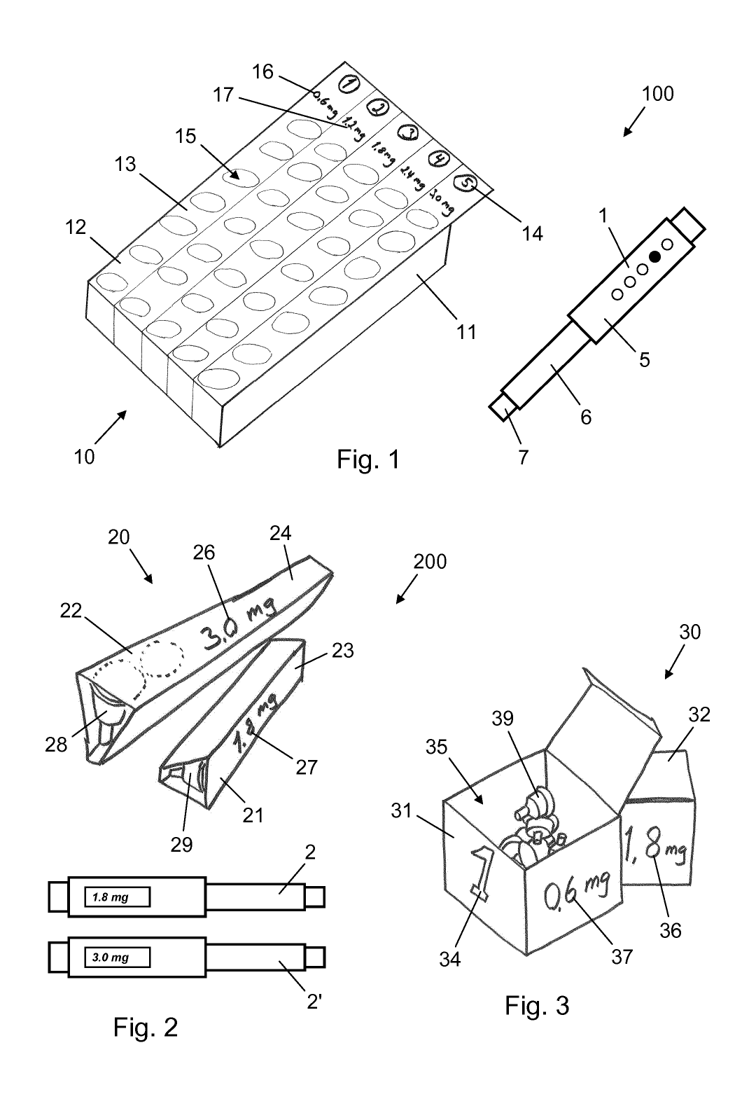

FIG. 1 shows a dose titration system 100 according to a first embodiment of the invention. The dose titration system 100 comprises an injection device 1 and a needle box 10. The injection device 1 comprises dose setting and injection mechanisms which are accommodated in a housing 5, and is capable of delivering five predetermined dose sizes of a drug from an exchangeable drug cartridge (not visible) held in a cartridge holder 6. A needle interface 7 is provided at the distal end of the injection device 1 for reception of an injection needle (not shown).

The needle box 10 comprises a case 11 and a top cover 12. The top cover 12 is visually divided into five columns 13, and each column 13 is provided with a respective number 14 indicating a position in a sequence. Further, each column 13 has seven needle wells 15, each needle well 15 being structured to contain one sealed injection needle (not shown), as well as a specific dose size indication 16, 17.

The dose titration system 100 is intended for use as follows. The needle box 10 initially holds one sealed injection needle in each of the 35 needle wells 15. When a user starts a dose titration period the first injection needle to be used is taken from the column numbered “1”. This injection needle is mounted on the needle interface 7 of the injection device 1 and the injection device 1 is set to deliver a dose corresponding to the dose size indication 16, i.e. a dose of 0.6 mg. After dose administration the injection needle is discarded and the needle interface 7 is thus ready to receive a new injection needle. For the next dose administration the user chooses another injection needle from column “1”, mounts it on the needle interface 7, and operates the injection device 1 to deliver another dose of 0.6 mg.

After seven injections and use of seven injection needles column “1” is empty and the user must select an injection needle from column “2” for the following dose administration. In doing that, the user is alerted to the fact that the second dose titration step has been reached and, accordingly, that the injection device 1 must be set to deliver a dose corresponding to the dose indication 17, i.e. a dose of 1.2 mg. The sketched procedure is continued for the remainder of the dose titration period.

The above described dose titration system 100 is i.a. useful for once daily dosing over a period of five weeks. The dose size associated with column “5” may be the target dose for subsequent regular administration.

FIG. 2 shows a dose titration system 200 according to a second embodiment of the invention. The dose titration system 200 functions in principle much like the dose titration system 100 according to the first embodiment of the invention. However, instead of a single injection device which is able to deliver a plurality of predetermined dose sizes, the dose titration system 200 comprises two fixed dose injection devices 2, 2′ which are each able to deliver a single predetermined dose size. The dose titration scheme to be followed with this system consists of a first number of administrations of 1.8 mg and a second number of administrations of 3.0 mg. Accordingly, the dose titration system 200 further comprises a needle casing 21 carrying the first number of injection needles 29 on a substrate 23, and another needle casing 22 carrying the second number of injection needles 28 on a substrate 24. The needle casing 21 comprises a dose size indication 27 of 1.8 mg and the needle casing 22 comprises a dose size indication 26 of 3.0 mg. The injection needles 29 carried in needle casing 21 are for use with the injection device 2, which is structured to deliver 1.8 mg of a contained drug each time an accommodated injection mechanism is activated. Similarly, the injection needles 28 carried in needle casing 22 are for use with the injection device 2′, which is structured to deliver 3.0 mg of its contained drug each time an accommodated injection mechanism is activated.

FIG. 3 shows a needle arrangement 30 for use in a dose titration system, e.g. of the type described in the above. The needle arrangement 30 comprises a needle box 31 holding a number of injection needles 39 in an interior space 35, and a further needle box 32, similarly holding a number of injection needles (not visible). The needle box 31 has a dose size indication 37 of 0.6 mg printed on one exterior surface and a sequence related indication 34 printed on a second exterior surface, informing the user that the injection needles 39 in that particular box are to be used in the first dose titration phase when delivering a dose of 0.6 mg. The needle box 32 has a dose size indication 36 of 1.8 mg printed on one exterior surface and a sequence related indication (not visible) printed on a second exterior surface, informing the user that the injection needles in that box are to be used in the second dose titration phase when delivering a dose of 1.8 mg.

1. A needle arrangement comprising:
a plurality of injection needles,
one or more needle containers for the plurality of injection needles, each of the one or more needle containers being structured to hold at least one of the plurality of injection needles, and
a dedication of each of the plurality of injection needles held in the one or more needle containers to one of at least two predetermined dose sizes.

2. A needle arrangement according to claim 1, wherein the dedication of each of the plurality of injection needles held in the one or more needle containers to one of at least two predetermined dose sizes comprises a representation of the one of the at least two predetermined dose sizes.

3. A needle arrangement according to claim 2, wherein the representation of the one of the at least two predetermined dose sizes comprises a number.

4. A needle arrangement according to claim 3, wherein the representation of the one of the at least two predetermined dose sizes further comprises a unit.

5. A needle arrangement according to claim 1, wherein the plurality of injection needles are arranged in groups, each group consisting of injection needles with identical dedication.

6. A needle arrangement according to claim 1, wherein the plurality of injection needles comprises a first number of injection needles corresponding to a first prescribed number of injections of a first predetermined dose size, and a second number of injection needles corresponding to a second prescribed number of injections of a second predetermined dose size.

7. A needle arrangement according to claim 1, wherein the one or more needle containers comprise a plurality of predefined positions for the plurality of injection needles, and wherein each of the plurality of predefined positions is a) adapted to hold one injection needle, and b) associated with a dose related marking.

8. A needle arrangement according to claim 7, wherein the plurality of predefined positions are arranged in arrays, each array holding a portion of the plurality of predefined positions which corresponds to a prescribed number of injections of one of the at least two predetermined dose sizes.

9. A needle arrangement according to claim 8, wherein each array holds seven of the plurality of predefined positions.

10. A needle arrangement according to claim 7, wherein each of the plurality of predefined positions is a well configured to contain one injection needle.

11. A needle arrangement according to claim 6, wherein the one or more needle containers comprise a first box adapted to hold the first number of injection needles and comprising a representation of the first predetermined dose size, and a second box adapted to hold the second number of injection needles and comprising a representation of the second predetermined dose size.

12. A needle arrangement according to claim 1, wherein the one or more needle containers comprise an indication of the sequence in which the at least two predetermined dose sizes of the injectable drug are to be administered.

13. A system for administering at least two predetermined dose sizes of an injectable drug, the system comprising:
one or more drug delivery devices, each of the one or more drug delivery devices being capable of delivering one or more of the at least two predetermined dose sizes of the injectable drug, and
a needle arrangement according to claim 1.
